(19) United States
(12) Patent Application Publication
(10) Pub. No.: US 2018/0214762 A1
(43) Pub. Date: Aug. 2, 2018

(54) PROTECTIVE AND HEALING INNER CHEEK PADS

(71) Applicant: Samuel Aminia, Los Angeles, CA (US)

(72) Inventor: Samuel Aminia, Los Angeles, CA (US)

(21) Appl. No.: 15/805,208

(22) Filed: Nov. 7, 2017

Related U.S. Application Data
(60) Provisional application No. 62/451,861, filed on Jan. 30, 2017.
(60) Provisional application No. 62/451,853, filed on Jan. 30, 2017.

(51) Int. Cl. A63B 71/08; A61C 19/06

(52) U.S. Cl. CPC A63B 71/085; A61F 5/566; A61C 19/063

(57) ABSTRACT

The present invention recognizes that there is a long felt need for devices that can prevent self-inflicted or other types of wounds to the inner surfaces of the mount, in particular the inner cheek, and to promote the healing of such wounds. A first aspect of the present invention generally relates to a protective inner cheek pad. A second aspect of the present invention generally relates to a method of making a protective inner cheek pad. A third aspect of the present invention generally relates to a method of using a protective inner cheek pad. A fourth aspect of the present invention generally relates to a medicated inner cheek pad. A fifth aspect of the present invention generally relates to a method of making a medicated inner cheek pad. A sixth aspect of the present invention generally relates to a method of using a medicated inner cheek pad.

### RELATED APPLICATIONS AND PRIORITY STATEMENT

The present application claims benefit of priority to U.S. Provisional Application Ser. No. 62/451,861 filed Jan. 30, 2017, and U.S. Provisional Application Ser. No. 62/451,853 filed Jan. 30, 201, each of which are incorporated by reference herein in their entirety for all purposes.

### TECHNICAL FIELD

The present invention relates generally to the field of intra-oral devices and methods of making and methods of using of same that protect the inner surfaces of the mouth from damage. In particular, the devices of the present invention protect the inner cheek, particularly from single or repeated self-inflicted biting such as but not limited to from habit or after dental procedures. In addition, the present invention relates to the healing and enhanced healing of such wounds. Furthermore, the present invention relates to drug delivery device and method of making and method of using same.

### BACKGROUND

There are a variety of ways that the inside cheek surface, or other surfaces within the buccal cavity, can become injured. These include, but are not limited to: 1) biting cheek while eating due to careless chewing, 2) wisdom teeth (molars) biting into cheek due to teeth misalignment, 3) cheek biting habit due to anxiety and nervous habits, 4) cheek biting due to talking while eating, 5) compulsive cheek biting (which can require therapy), 6) biting teeth in sleep (like teeth grinding has to do with daily stress), 7) Buccarum Morsicatio is a condition characterized by chronic cheek biting and chronic cheek chewing, and 8) biting after dental or other sedation.

As a general example of causation of cheek biting, while chewing on a hard food, or while chewing gum, or in an uncontrolled jaw movement, the inner cheeks are bitten by teeth causing a painful cut or a bite mark, The resulting bite mark should take only 2 to 4 days to heal, however often times it takes a longer than necessary to heal mainly due to repeated bites by the teeth in the same area. One main reason for repeated bites is that the inner cheek area is swollen from the initial bite incident.

In some instances, such a bite or series of bites results in a more severe ulcer or infection which in turn takes even longer to heal. This becomes a bigger problem for adults since adults have more involuntary jaw movement. In addition, for older adults, it takes longer time for any wounds to heal. Also, as mentioned by the American Diabetes Association, people with diabetes are more prone to gum disease and oral fungal infections and therefore they heal more slowly from oral and dental surgery and other oral discomforts.

As an additional general example of causation of cheek biting is when dental work is done on a patient's teeth, often times the gums and cheeks are numbed or sedated by numbing medication. From the time the dental work is completed, it takes several hours for patients to fully regain feeling in the gums and cheeks. During this time, there is a danger of biting into the cheeks without feeling the pain and hence causing injury or damage to the inner cheeks. This problem affects both children and adults. To prevent this accidental biting, a device of the present invention can be temporarily placed between the teeth and the inner cheeks until normal feeling is regained in the inner cheek area. Preferably, after a dental or oral procedure, the dentist or the orthodontist would provide a device of the present invention to the patient to take home.

A further general example of causation of cheek damage is when braces are placed on the teeth following an orthodontic procedure, very often, the brace wires become loose or dislodged or partially break off. Sometimes the brackets reveal a sharp tip that can push against cheeks and gums and cause injury, pain, and discomfort. This always requires a revisit to the orthodontist so the braces can be repaired. The present invention provides a solution to the above problems by providing a temporary barrier between the sharp parts of the broken braces and the cheeks and gums. This barrier is helpful in temporary prevention of any injury or any further injury to the inner cheeks until a new appointment has been scheduled with the orthodontist and the braces are repaired. Preferably, at the time new braces are placed for the first time, the orthodontist would provide a device of the present invention, or several of them, to the patient to take home. This is a preventive measure based on the fact that nearly all braces will break or be dislodged at some point in the course of their use while in the patient's mouth.

Generally, the present invention provides a solution to the above problems relating to biting of inner mouth parts, notably but not limited to the inner cheek, by providing a barrier between the teeth and the inner cheeks to prevent bites and subsequently speed up the healing time of bite marks and canker sores by preventing repeated bites.

The present invention is intended to protect and to prevent injury to the inner cheek area of the mouth. In addition, the present invention is intended to sooth, bring comfort to, and bring about speedy healing of the inner cheeks of the mouth after an injury has occurred.

Generally, depending on the circumstances, a device of the present invention can be medicated or not medicated. When medicated, a device of the present invention can include a medicament for a desired disease, disorder, or condition in a deliverable form. Medicated and non-medicated versions of a device of the present invention can be used for preventative purposes, treatment purposes, or a combination thereof.

Finally, an additional aspect of the present invention is to assist with the delivery of medication through saliva or otherwise within the buccal cavity or by swallowing using a medicated version of the present invention, In certain situations, medications are needed to be administered thru saliva in the cheeks as opposed to being ingested thru the mouth and stomach by way of the digestive system. Examples of patients or subjects in need of such devices of the present invention include but are not limited to: 1) subjects who have a hard time swallowing pills, 2) subjects with anxiety issues which cause phobia of swallowing pills, 3) subjects with acute gag reflex which can cause vomiting, 4) subjects with certain liver conditions, 5) subjects with various digestive system problems, 5) subjects following a stroke, 6) subjects having surgery, 7) subjects having gastroesophageal reflux (GERD), 8) subjects with neurological disorders (such as but not limited to multiple sclerosis, Parkinson's disease, Alzheimer's disease, and the like). The medicated versions of the devices of the present invention address these issues, and provide related benefits as well.

### SUMMARY

The present invention recognizes that there is a long felt need for devices that can prevent self-inflicted or other types of wounds to the inner surfaces of the mount, in particular the inner cheek, and to promote the healing of such wounds.

A first aspect of the present invention generally relates to a protective inner cheek pad.

A second aspect of the present invention generally relates to a method of making a protective inner cheek pad.

A third aspect of the present invention generally relates to a method of using a protective inner cheek pad.

A fourth aspect of the present invention generally relates to a medicated inner cheek pad.

A fifth aspect of the present invention generally relates to a method of making a medicated inner cheek pad.

A sixth aspect of the present invention generally relates to a method of using a medicated inner cheek pad.

### BRIEF DESCRIPTION OF THE FIGURES

FIG. 1A generally depicts one aspect of a device of the present invention, large adult size, operably engaged in the mouth of an adult male subject. The dashed lines refer to what is present but not seen, and is presented in solid lines in FIG. 1B. FIG. 1B generally depicts one aspect of a device of the present invention as located between the inner cheek and outer surface of the teeth and gums and related surfaces and structures of the subjects buccal cavity. Note where the device is located relative to the teeth, which is described in greater detail in later figures. Elements in the figures are as follows: 150 is a subject; 100 is a device of the present invention; 110 is the upper jaw; 120 is the lower jaw; 130 are the upper teeth; 140 are the lower teeth; and 160 is the tongue.

FIG. 2 generally depicts one aspect of a device of the present invention, large adult size, operably engaged in the mouth of an adult male subject, Note where the device is located relative to the teeth, which is described in greater detail in later figures. Elements in the figures are as follows: 210 is the upper lip; 220 is the lower lip; 230 is buccal cavity; and 240 is the inner cheek.

FIG. 3A generally depicts one aspect of a device of the present invention, large adult size, operably engaged in the mouth of an adult male subject. The dashed lines refer to what is present but not seen, and is presented in solid lines in FIG. 3B. FIG. 3B generally depicts one aspect of a device of the present invention as located between the inner cheek and outer surface of the teeth and gums and related surfaces and structures of the subjects buccal cavity. Note where the device is located relative to the teeth, which is described in greater detail in later figures. Note that these figures FIG. 3A and FIG. 3B are similar to FIG. 1A and FIG. 1B but have a different view of the subject and the positioning of the jaws being open rather than closed.

FIG. 4 generally depicts one aspect of a device of the present invention, youth size, being operably engaged, in other words in the process of being inserted, into the mouth of a youth female. Elements in the figures are as follows: 410 is the hard palate

FIG. 5A, FIG. 5B, FIG. 5C, and FIG. 5D generally depict one aspect of a device of the present invention, a large adult size. FIG. 5A is a top and bottom view, FIG. 5B is a side view, FIG. 5C is a perspective view; and FIG. 5D depicts the device in a flexed state. Elements in the Figures are as follows: 500 is a first planar structure; 510 is a second planar structure; and 520 is a circumference surface.

FIG. 6A, FIG. 6B, FIG. 6C, and FIG. 6D generally depict one aspect of a device of the present invention, a small adult size. FIG. 6A is a top and bottom view, FIG. 6B is a side view, FIG. 6C is a perspective view; and FIG. 6D depicts the device in a flexed state.

FIG. 7A, FIG. 7B, and FIG. 7C generally depict one aspect of a device of the present invention, a youth size. FIG. 7A is a top and bottom view, FIG. 7B is a side view, and FIG. 7C is a perspective view.

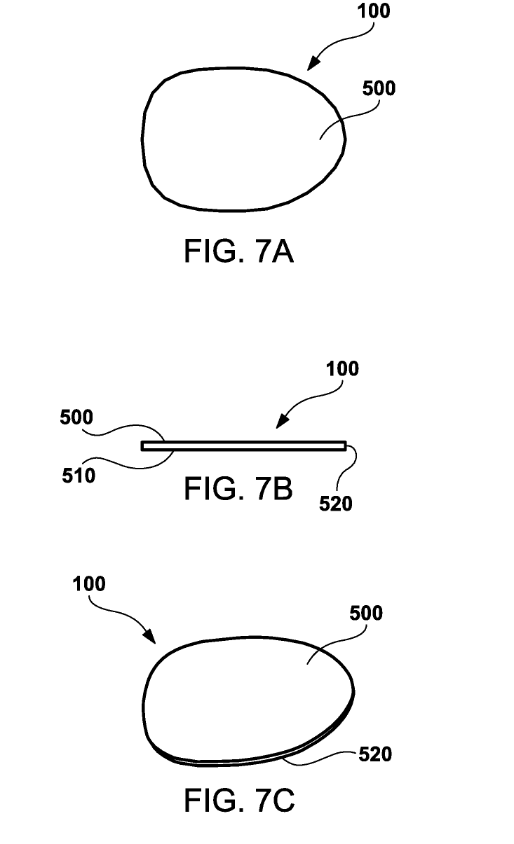

FIG. 8 generally depicts one aspect of a device of the present invention, a medicated inner cheek pad. In this aspect of the present invention, a medicated strip or tab that can release a medicament or other substance is provided on the surface of a device of the present invention. Elements in the figures are as follows: 800 is a medicated inner cheek pad, and 810 is a medicated layer.

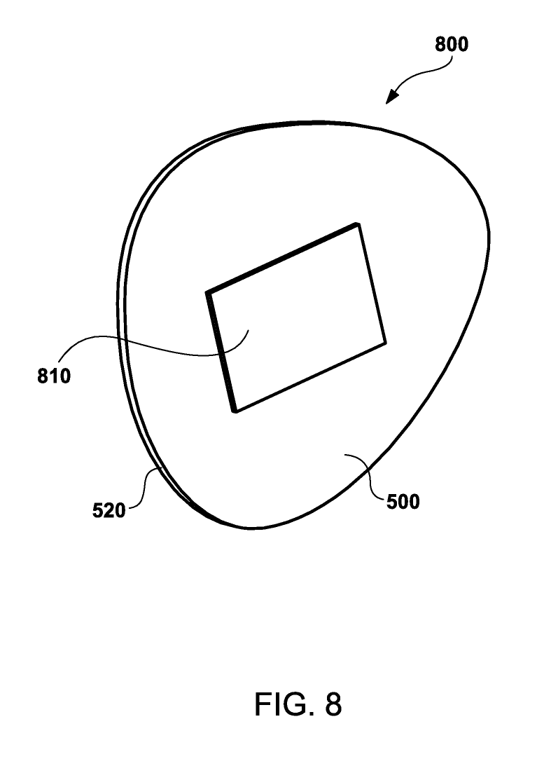

FIG. 9 generally depicts the mouth parts of a subject, highlighting the teeth and soft tissues and bone to depict which structures a device of the present invention operably engages with a subject. Elements in the figures are as follows: 910 is the floor of mouth; 920 are molars; 930 are premolars; 940 is the canine tooth; 950 are incisors; 960 are upper gums; 970 are lower gums; and 980 is the soft palate. Preferably, a device of the present invention, when operably engaged with a subject, is in contact with any of the teeth 920, 930, 940, and 950. More preferably a device of the present invention, when operably engaged with a. subject, is in contact with any of the teeth 920, 930, and 940. Still more preferably a device of the present invention, when operably engaged with a subject, is in contact with any of the teeth 920 and 930. A device of the present invention when operably engaged with a subject is also in contact with the inner cheek, upper gums, and lower gums, either alone or in combination at any particular point of time when the device is in use.

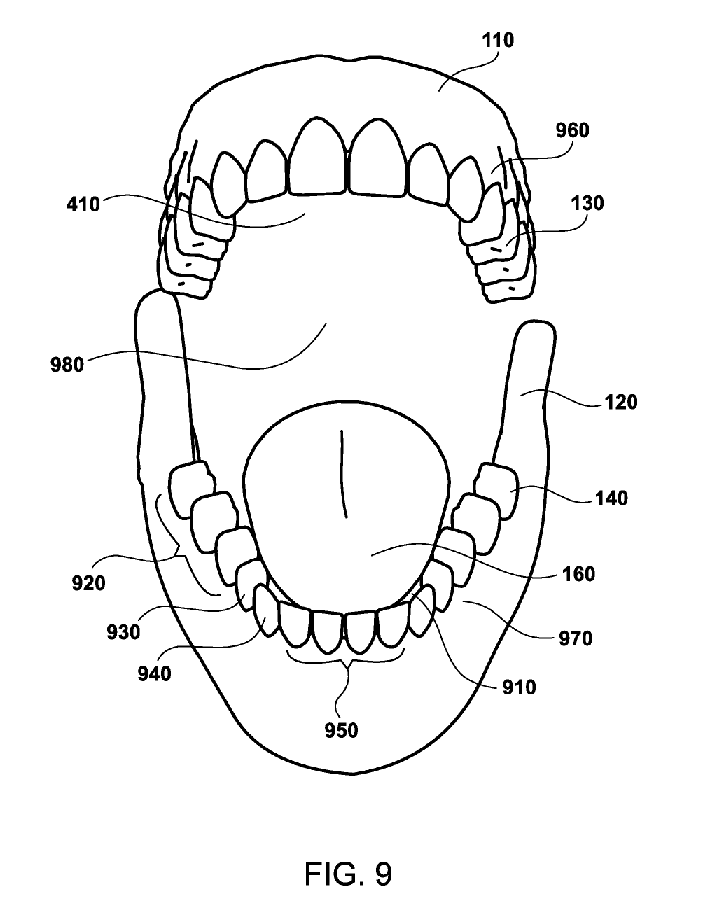

FIG. 10 generally depicts the mouth parts of a subject, highlighting the teeth and soft tissues to depict which structures a device of the present invention operably engages with a subject. Elements in the figures are as follows: 1000 is the hard palate; 1010 is the soft palate; 1020 is the uvula; 1030 is a palatine tonsil; 1040 is the gingiva or gums; 1050 is the sublingual area; 1060 is the inner cheek; 1070 back of throat; 1071 is the second molar; 1072 is the first molar; 1073 is the second bicuspid; 1074 is the first bicuspid; 1075 is the cupid or canine; 1076 is the lateral incisor; 1077 is the central incisor; and 1080 is the body of the tongue. A device of the present invention can be in contact with any of these structures while operably engaged with a subject. Preferably, in contact with any of the teeth and the inner cheek and gums. More preferably at least one molar, at least one bicuspid, and at least one cuspid along with the inner cheek and upper and lower gums on that side of the mouth. Still more preferably at least one molar, at least one cuspid, along with the inner cheek and upper and lower gums on that side of the mouth.

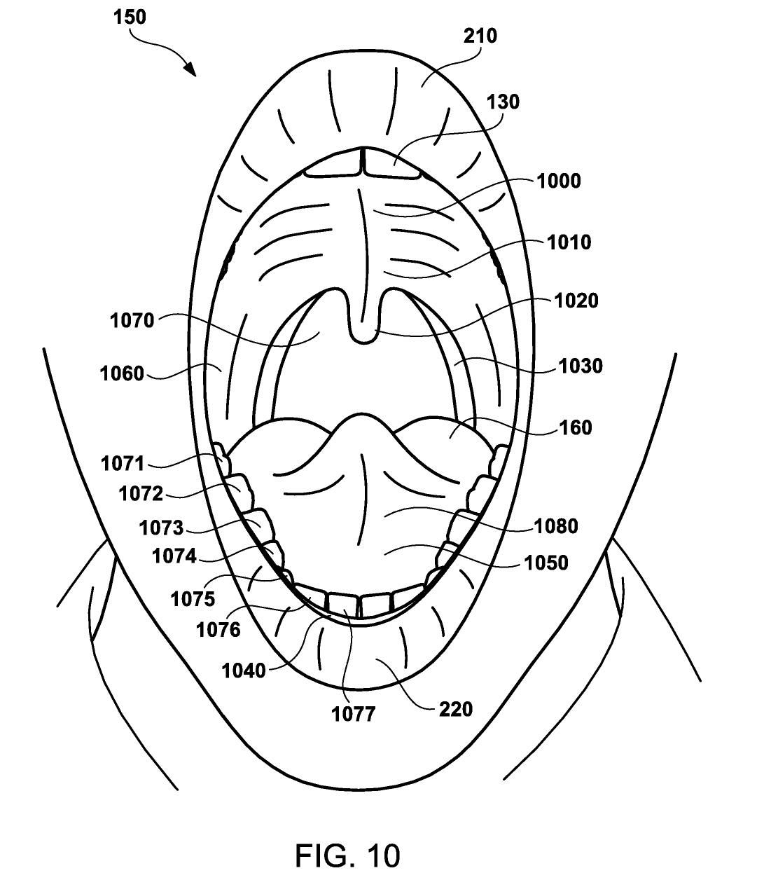

FIG. 11 generally depicts dyes to cut devices of the present invention from sheets of material, such as but not limited to medical or food grade silicone. The upper device shows the size and shape of large adult size with four individual dyes that can be provided separately or linked together into a unit. The lower devices shows the size and shape of youth size with four individual dyes that can be provided separately or linked together into a unit, Elements in the figures are as follows: 1100 is a dye for adult large size device; 1110 is a dye for youth size device; 1120 is a cutting edge of the dye; 1130 is a side of a dye; and 1140 is a top edge that can be cutting or not cutting.

### DETAILED DESCRIPTION OF THE INVENTION

### Definitions

Unless defined otherwise, all technical and scientific terms used herein have the same meaning as commonly understood by one of ordinary skill in the art to which this invention belongs, Generally, the nomenclature used herein and the laboratory procedures in cell culture, chemistry, microbiology, molecular biology, cell science and cell culture described below are well known and commonly employed in the art, Conventional methods are used for these procedures, such as those provided in the art and various general references such as Wikipedia, Healthline newsletter, Healthline Networks, Inc. Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

“Buccal cavity” refers to the portion of the oral cavity lying between the teeth and cheeks. Also referred to as the “vestibule of the mouth.”

“Buccal space” refers to a facial space of the head and neck, being the potential space in the cheek, and is paired on each side.

“Buccal mucosa” refers to the inner lining of the cheeks and floor of the mouth, including sub-lingual, and is a part of the lining mucosa.

“Buccal medicine administration” refers to the placement of the drug between the gums and the cheek, and on in close proximity to buccal mucosa, inclusive of sub-lingual administration. These medications can come in the form of tablets, films, sprays or otherwise known in the art.

“Buccal administration” refers to a topical route of administration by which drugs are held or applied in the inner cheek, inner mouth, or sub-lingual area, or other areas having buccal mucosa. These drugs diffuse thru the oral mucosa and enter directly into the bloodstream. The cheek area has many capillaries, or tiny blood vessels. There, drugs can be absorbed directly into the bloodstream without going thru the digestive system.

“Buccal drugs” refers to drugs that preferably absorb quickly by way of buccal administration. Since they don't go thru the digestive system, they are not metabolized thru the liver by the first pass effect and therefore lower dosages can get the same or similar results.

“Dysphagia condition” refers to a condition that causes difficulty or discomfort in swallowing, as a symptom of a disease.

Other technical terms used herein have their ordinary meaning in the art that they are used, as exemplified by a variety of technical dictionaries.

### Introduction

The present invention recognizes that there is a long felt need for devices that can prevent self-inflicted or other types of wounds to the inner surfaces of the mount, in particular the inner cheek, and to promote the healing of such wounds.

As a non-limiting introduction to the breath of the present invention, the present invention includes several general and useful aspects, including:

1) a protective inner cheek pad;

2) a method of making a protective inner cheek pad;

3) a method of using a protective inner cheek pad;

4) a medicated inner cheek pad;

5) a method of making a medicated inner cheek pad; and

6) a method of using a medicated inner cheek pad.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

### I. A Protective Inner Cheek Pad

The present invention includes as a first aspect a protective inner cheek pad device, including:

a) a flexible, thin, and planar structure configured to fit between the outer surface of the teeth of a subject and the inner surface of the cheek of a subject when in use in a subject;

b) the solid, thin, and planar structure having two planar surfaces and a circumference surface;

c) a first planar surface that is not liquid absorbent and can operably engage the outer surface that is of the teeth of a subject;

d) a second planar surface that is not liquid absorbent and can operably engage the inner surface of the cheek of a subject; and

wherein said inner cheek pad device prevents said subject from biting said inner surface of the cheek of said subject when operably engaged in the mouth of said subject; and

wherein said inner cheek pad device prevents said subject from injuring said inner surface of the cheek of said subject when operably engaged in the mouth of said subject when the subject has dental appliances engaged with said outer surface of the teeth of a subject.

The flexible, thin, and planar structure can be made of appropriate materials that conform to these characteristics, such as but not limited to those described herein and are known in the art. Preferred examples are polymers and plastics, but the present invention is not limited to those materials.

The not liquid absorbent characteristic of the materials is as is known in the art and described herein that conform to that characteristic. The not liquid absorbent characteristic refers to the material not substantially absorbing saliva or other water based liquids, notably so as the material retains its shape and does not compress or otherwise fold upon itself due to the absorption of such a liquid.

### A. Structure and Materials of Device

In another aspect of the present invention, the flexible, thin, and planar structure is a laminated structure, a not laminated structure, or a combination thereof. A device of the present invention is preferably thin and designed to fit within the confines of the buccal cavity of a subject, preferably between the outer surface of the teeth and the inner surface of the cheek of the subject, a space that does not have a substantial amount of comfortable room. A device of the present invention can be of a single thickness of material, or of multiple layers of material. The multiple layers of material can be a laminate and made using methods and materials as are known in the art based on the selection of materials for a device of the present invention.

In a further aspect of the present invention, the first planar surface and the second planar surface are made of the same material, different material, or a combination thereof. In addition to being a laminate, a device of the present invention can be of the same or different materials. If different materials are used, the materials can be in a laminate configuration (vertical orientation) or in side by side configuration (horizontal orientation), or a combination thereof. There can be two or more layers of laminated materials in the vertical orientation, horizontal orientation, or a combination thereof. Materials and methods used to make a device of the present invention in these configurations can be those know in the art and based on the selection of materials.

In an additional aspect of the present invention, the first planar surface, the second planar surface, or a combination thereof comprise non-toxic material. As a device of the present invention is to be place with the buccal cavity of a subject, a device of the present invention is preferable made of non-toxic materials. Those materials can be selected based on the non-toxic and other properties of materials based on the desired characteristics of a device of the present invention as set forth herein and as are known in the art.

In another aspect of the present invention, the first planar surface, the second planar surface, or a combination thereof comprise solid material, semi-solid material, or a combination thereof. Solid materials such as plastics and polymers are known in the art, as are semi-solid materials such as polymers and hydrogels that allow a certain amount of compression and “give” to a device of the present invention as well as holding additional components, such as but not limited to medicaments, which can be released from a device of the present invention. Semi-solid can also provide a level of comfort for the user and a device of the present invention can confirm more readily to the contours of the teeth and cheek of the user during operation. A device of the present invention in whole or in part can be not water soluble so as to not dissolve in the mouth, in whole or in part water soluble so as to have a limited useful live in situ and have the capability to deliver substances such as medicaments. Also, a hybrid device of water soluble and not water soluble components are part of the present invention.

In a further aspect of the present invention, the first planar surface, the second planar surface, or a combination thereof include plastic, rubber, polymer, or a combination thereof. These components can be used in whole or in part to make a device of the present invention. Preferably, the materials are selected based on the desired characterizes of a device of the present invention as described herein, and in accordance with the state of the art.

In an additional aspect of the present invention, the first planar surface, the second planar surface, or a combination thereof include silicone, medical grade silicone, food grade silicone, natural rubber, or a combination thereof. These materials are preferred materials for use in the manufacture of a device of the present invention.

In another aspect of the present invention, the first planar surface, the second planar surface, or a combination thereof comprise at least one not collapsible material. Not collapsible materials, such as those described herein, hold their shape under storage conditions, and in use conditions, for a device of the present invention. It is desired that a device of the present invention not fold in upon itself while in sure or when stored, particularly when in use as a device of the present invention would function for longer periods of time when not collapsible materials are used. A majority of the materials discussed herein would be considered not collapsible, inclusive of a majority of plastics, polymers, and other such materials.

In a further aspect of the present invention, the first planar surface, the second planar surface, or a combination thereof have the same texture, different texture, or a combination thereof. in some instances, the surface of a device of the present invention can be smooth, and in other instances the surface of a device of the present invention can be textured. Smooth surfaces can be more comfortable for the subject, for example. Rough or textured surfaces can, for example, enhance the ability of a device of the present invention to stay in place during operation.

In an additional aspect of the present invention, the first planar surface, the second planar surface, or a combination thereof include cushioned material, not cushioned material, or a combination thereof. Cushioned materials have been discussed herein and can include silicone, rubber and other compressible materials such as hydrogels and as they are known in the art. Cushioned materials can be desirable for the comfort of the user.

In another aspect of the present invention, the first planar surface, the second planar surface, or a combination thereof include a unitary structure, a non-unitary structure, or a combination thereof. A device of the present invention can be provided, and used, as a single unit or as multiple units.

### B. Shape of Device

In another aspect of the present invention, the solid, thin, and planar structure is generally triangular, generally square, generally polygonal, generally circular, generally oval or a combination thereof, in shape. In general, a device of the present invention can be of any shape and size that is appropriate. These are but a few of the possible configurations of a device of the present invention. The size of a device of the present invention can be in the range of about 30 mm×about 30 mm with a thickness of about 1.0 mm; and about 45 mm×about 55 mm with a thickness of about 2.0 mm. Preferred sizes are about 35 mm×about 40 mm with a thickness of about 1.5 mm; and about 38 mm×about 44 mm with a thickness of about 1.5 mm. These sizes are preferable for a human subject having a normal sized buccal cavity, and can be changed as appropriate or needed for a human subject of different sized buccal cavity or for a non-human subject.

In a further aspect of the present invention, the solid, thin, and planar structure has rounded corners. Rounded corners, when corners are present, would tend to provide enhanced comfort for the subject when a device of the present invention is in use. Thus, this configuration is a preferred aspect of the present invention.

In an additional aspect of the present invention, the solid, thin, and planar structure has one or more through holes, indentations, partial through holes, or a combination thereof to allow release of components within said device, allow fluids to pass through said device, enhance the comfort of a device of the present invention, assist in said device placement and staying in place, or a combination thereof. Through holes can be made by molding, drilling, cutting, laminating, and the like and provide through space so that saliva and other fluids can readily pass through a device of the present invention to add comfort for the subject when a device of the present invention is in place, and can enhance the release of materials such as medicament when present within or on a device of the present invention. Partial through holes, like dimples on a golf ball, can be made in the same way as through holes. These partial through holes can be used to expose internal layers of a device of the present invention, such as those including medicaments that can be released from a device of the present invention.

### C. Medicament

In another aspect of the present invention, a device of the present invention includes at least one medicament in a pharmaceutically effective amount to treat a disease, disorder or condition of a subject when said device operably engaged in the buccal cavity of said subject. Any appropriate medicament can be used. Preferably, the medicament is used to treat wounds of the mouth, particularly those in contact with or in close proximity to a device of the present invention when in use with a subject, but that need not be the case.

In a further aspect of the present invention, the medicament is within said device, on the surface of said device, or a combination thereof. A medicament can be provided in an appropriate amount within a device of the present invention, such as in a drug containing layer internal to a device of the present invention, or can be provided in an outer layer of a device of the present invention, or throughout a device of the present invention. Alternatively, an additional structure, such as a tab or strip of drug containing material can be used, Such tabs or strips can be readily dissolvable or slowly dissolvable while in use with a subject.

In an additional aspect of the present invention, the medicament comprises a drug, a therapeutic, a remedy, a soothing agent, a pain reliever, an anti-inflammatory, and anti-infective, and anti-bacterial, and antibiotic, and anti-viral, an anti-fungal, anti-yeast, or a combination thereof. This list of medicaments is not limiting and relates to the types of diseases, disorders, and conditions that can be expected in the buccal cavity of a subject when a device of the present invention is in use. For example, soothing agents can be used to treat cold sores or tooth aches, and anti-yeast agents can be used to treat thrush and other conditions. The selection of the agents and dose thereof can be a design choice of the skilled artisan, particularly looking towards the dose, route of administration, and regime of administration. The positioning of a device of the present invention adjacent to or in close proximity to the locus of disease, disorder, or condition of the subject can reduce the required overall dose provided by a device of the present invention and released by a device of the present invention while in operation.

In another aspect of the present invention, a device of the present invention is operably engaged in the buccal cavity of a subject, said device releases a pharmaceutically effective amount of said medicament to be absorbed by the oral mucosa, to be swallowed, or a combination thereof. The medicament released by a device of the present invention can be intended to be delivered to the point of contact of a device of the present invention within the buccal cavity, areas in close proximity to a device of the present invention, the entire buccal cavity, to be absorbed through the mucosa. In the alternative, the medicament can be intended to swallowed and delivered to the gastrointestinal tract. Or, a combination of absorption and swallowing. Drugs absorbed through the oral mucosa, including sublingual, does not pass directly to the liver for detoxification of the drug as is the case when drugs are absorbed in the digestive tract, and thus in the instance of absorption within the buccal cavity, less drug can be expected to be administered.

In a further aspect of the present invention, the medicament treats a disease, disorder, or condition of said subject. The selection and choice of drug or drugs to be administered by way of a device of the present invention, the dose thereof, the route of administration, the regime, and other routine pharmacological factors depends on the disease, disorder, or condition of the subject, and the locus of that disease, disorder, or condition and related pharmacological considerations, being within the skill of the ordinary artisan. The variables and factors are combined to provide an efficacious treatment for a disease, disorder, or condition of a subject using a device of the present invention while operably engaged with a subject.

### D. Subject

In another aspect of the present invention, the subject can be a mammal, a non-human primate, or a human. Preferably the subject is a human, being of any age, preferably having at least one tooth.

### II. A Method of Making a Protective Inner Cheek Pad

The present invention includes as a second aspect a method of making a protective inner cheek pad device of the present invention, including:
a) in the instance of molding manufacture, comprising the following steps:
   i) provide moldable polymer;
   ii) molding said device; and
   iii) optionally performing finishing for said device;
b) in the instance of laminating manufacture, comprising the following steps:
   i) provide multiple sheets of material;
   ii) laminating said multiple sheets of material together for form a laminate;
   iii) optionally cutting out or otherwise removing devices from said laminate; and
   iv) optionally performing finishing for said device; and
c) in the instance of single layer manufacture, comprising the following steps:
   i) providing a sheet of material;
   ii) cutting out or otherwise removing devices from said sheet of material; and
   iii) optionally performing finishing for said device;
d) a combination of a), b), and c);
wherein a device of the present invention is produced,

In general, a device of the present invention can be made using any appropriate materials, any appropriate means, and any appropriate methods of manufacture as are known in the art or later developed. Some of those materials, means, and methods are described herein.

In one aspect of the present invention, a device of the present invention can be made in whole or in part by 3D printing. 3D printing is known in the art, and the section of materials, methods and the like to manufacture a device of the present invention can be readily accomplished based on the state of the art.

In another aspect of the present invention, the molding in a) above includes injection molding. Injection molding is well known in the art, and the section of materials, methods and the like to manufacture a device of the present invention can be readily accomplished based on the state of the art.

In a further aspect of the present invention, the laminating in b) above includes heat welding, melting, partial melting, sonic welding, adhesives, or a combination thereof. These methods of adhering surfaces together, such as but not limited to those made of plastic or polymer, are well known in the art, and the section of materials, methods and the like to manufacture a device of the present invention can be readily accomplished based on the state of the art.

In another aspect of the present invention, the cutting out or otherwise removing devices in b) and c) above includes cutting devices, cutting methods, scissors, razors, dyes, cutting dyes, or a combination thereof. These methods of cutting materials, such as but not limited to those made of plastic or polymer, are well known in the art, and the section of materials, methods and the like to manufacture a device of the present invention can be readily accomplished based on the state of the art.

In an additional aspect of the present invention, the finishing in a), b), and c) above includes smoothing or roughening of surfaces, corners, edges, or a combination thereof. When considering the comfort of a user of a device of the present invention, the surfaces, corners, and edges of a device of the present invention can preferably be smooth. In order to have a device of the present invention stay in place while in use, those surfaces, corners, and edges may be roughened or remain rough. Smoothing techniques such as polishing are known in the art, such as by buffing or otherwise polishing by chemical means, mechanical means, or a combination thereof as they are known in the art. Surface roughening can be likewise accomplished by chemical means, mechanical means, or a combination thereof as they are known in the art.

In another aspect of the present invention, the finishing in a), b), and c) above includes rounding of surfaces, corners, edges, or a combination thereof. When considering the comfort of a user of a device of the present invention, the surfaces, corners, and edges of a device of the present invention can preferably be rounded. Rounding techniques such as polishing are known in the art, such as by buffing or otherwise polishing by chemical means, mechanical means, or a combination thereof as they are known in the art.

In a further aspect of the present invention, the finishing in a), b), and c) above includes adding at least one additional component to a core of a device of the present invention, Additional components, for example, can be layers of material can be added to a device of the present invention, and can be made of the same or different materials as the core device, such as by lamination or adhesion. The additional component can be the same shape and size as a core device of the present invention, or can be smaller or larger, and thus fit in size and/or dimension, be larger in size and/or dimension, smaller in size and/or dimension, or a combination thereof.

In an additional aspect of the present invention, the at least one additional component comprises at least one medicament. In one preferred aspect of the present invention, the additional component includes a drug. Further, the drug can provided in an additional layer. The additional layer with a drug can be, for example, in a tab form of dissolvable material, resorbable material, degradable material, or a combination thereof that releases drug when operably engaged in the buccal cavity of a subject when in operation. Or, the drug can be released from a layer that does not dissolve, resorb, or degrade in the buccal cavity of a subject when in operation.

### III. A Method of Using a Protective Inner Cheek Pad

The present invention includes as a third aspect a method of using a protective inner cheek pad device of the present invention, including:
a) providing a device of the present invention;
b) providing a subject in need of a device of the present invention; and
c) operable engaging a device of the present invention within the buccal cavity of the subject;
wherein the subject obtains the benefits of the device of the present invention.

These aspects of the present invention, along with others, are described in detail herein. Generally, a device of the present invention is operably engaged between the cheek and outer surface of the teeth within the buccal cavity of a subject, and the benefits described herein and below, and others as well, are realized by the subject.

In a further aspect of the present invention, the benefits of the device of the present invention include:
a) wherein the device of the present invention prevents the subject from biting the inner surface of the cheek of the subject when operably engaged in the mouth of the subject;
b) wherein the device of the present invention prevents the subject from injuring the inner surface of the check of the subject when operably engaged in the mouth of the subject when the subject has dental appliances engaged with the outer surface of the teeth of a subject;
c) wherein the device of the present invention provides medicaments to treat one or more wounds of the mouth; and
d) a combination thereof.

These aspects of the present invention, along with others, are described in detail herein. Generally, a device of the present invention when in use can prevent or treat biting of the inner surface of the cheek, prevent or treat other injury to the inner surface of the cheek, or a combination thereof. In addition, a device of the present invention can provide at least one medicament to the locus of such injury, or injury within the buccal cavity of the subject, or can be a route of administration for a medicament to be absorbed by the buccal cavity of said subject, inclusive of sub lingual, being swallowed to be absorbed by the gastro intestinal tract, or a combination thereof. Other benefits of the use of a device of the present invention are also provided and described here, or are otherwise provided.

In one preferred embodiment of the present invention, the is wherein the device of the present invention prevents the subject from injuring the inner surface of the cheek of the subject when operably engaged in the mouth of the subject, pediatric or adult, when the subject has dental appliances, namely orthodontic braces and the like, engaged with the outer surface of the teeth of the subject. In this instance, such braces are routinely damaged and sharp edges and points result that protrude and poke or otherwise damage the inside of the mouth, particularly the inner cheek of the user. Braces tend to be in place in a subject for about a year or more, and the instance of such breakage is essentially 100%. Currently, the wearer of braces is provided wax or other material to place over the broken braces, to use when such breakage occurs, which is not a very viable option. A device of the present invention can be provided to wearers of braces for use when discomfort from normal usage of braces, to prevent injury to the cheek or other areas of the mouth such as while taking part in sports or other activities including sleeping and such where braces can irritate the mouth, and when braces break and can and do result in injury to the inner cheek.

### IV A Medicated Inner Cheek Pad

The present invention includes as a fourth aspect a medicated inner cheek pad device, including:

a) a flexible, thin, and planar structure configured to fit between the outer surface of the teeth of a subject and the inner surface of the cheek of a subject when in use in a subject;

b) said solid, thin, and planar structure having two planar surfaces and a circumference surface;

c) a first planar surface that is not liquid absorbent and can operably engage the outer surface that is of the teeth of a subject;

d) a second planar surface that is not liquid absorbent and can operably engage the inner surface of the cheek of a subject; and

e) at least one medicament provided in a pharmaceutically effective amount within the device, on the surface of said device, or a combination thereof;

wherein when the device is operably engaged in the buccal cavity of a subject, the device releases a pharmaceutically effective amount of the medicament to be absorbed by the oral mucosa, to be swallowed, or a combination thereof; and

wherein said medicament treats a disease, disorder, or condition of the subject.

The flexible, thin, and planar structure can be made of appropriate materials that conform to these characteristics, such as but not limited to those described herein and are known in the art. Preferred examples are polymers and plastics, but the present invention is not limited to those materials.

The not liquid absorbent characteristic of the materials is as is known in the art and described herein that conform to that characteristic. The not liquid absorbent characteristic refers to the material not substantially absorbing saliva or other water based liquids, notably so as the material retains its shape and does not compress or otherwise fold upon itself due to the absorption of such a liquid.

### A. Structure and Materials of Device

In another aspect of the present invention, the flexible, thin, and planar structure is a laminated structure, a not laminated structure, or a combination thereof. A device of the present invention is preferably thin and designed to fit within the confines of the buccal cavity of a subject, preferably between the outer surface of the teeth and the inner surface of the cheek of the subject, a space that does not have a substantial amount of comfortable room. A device of the present invention can be of a single thickness of material, or of multiple layers of material. The multiple layers of material can be a laminate and made using methods and materials as are known in the art based on the selection of materials for a device of the present invention.

In a further aspect of the present invention, the first planar surface and the second planar surface are made of the same material, different material, or a combination thereof. In addition to being a laminate, a device of the present invention can be of the same or different materials. If different materials are used, the materials can be in a laminate configuration (vertical orientation) or in side by side configuration (horizontal orientation), or a combination thereof. There can be two or more layers of laminated materials in the vertical orientation, horizontal orientation, or a combination thereof. Materials and methods used to make a device of the present invention in these configurations can be those know in the art and based on the selection of materials.

In an additional aspect of the present invention, the first planar surface, the second planar surface, or a combination thereof comprise non-toxic material. As a device of the present invention is to be place with the buccal cavity of a subject, a device of the present invention is preferable made of non-toxic materials. Those materials can be selected based on the non-toxic and other properties of materials based on the desired characteristics of a device of the present invention as set forth herein and as are known in the art.

In another aspect of the present invention, the first planar surface, the second planar surface, or a combination thereof comprise solid material, semi-solid material, or a combination thereof. Solid materials such as plastics and polymers are known in the art, as are semi-solid materials such as polymers and hydrogels that allow a certain amount of compression and “give” to a device of the present invention as well as holding additional components, such as but not limited to medicaments, which can be released from a device of the present invention. Semi-solid can also provide a level of comfort for the user and a device of the present invention can confirm more readily to the contours of the teeth and cheek of the user during operation. A device of the present invention in whole or in part can be not water soluble so as to not dissolve in the mouth, in whole or in part water soluble so as to have a limited useful live in situ and have the capability to deliver substances such as medicaments. Also, a hybrid device of water soluble and not water soluble components are part of the present invention.

In a further aspect of the present invention, the first planar surface, the second planar surface, or a combination thereof include plastic, rubber, polymer, or a combination thereof. These components can be used in whole or in part to make a device of the present invention. Preferably, the materials are selected based on the desired characterizes of a device of the present invention as described herein, and in accordance with the state of the art.

In an additional aspect of the present invention, the first planar surface, the second planar surface, or a combination thereof include silicone, medical grade silicone, food grade silicone, natural rubber, or a combination thereof. These materials are preferred materials for use in the manufacture of a device of the present invention.

In another aspect of the present invention, the first planar surface, the second planar surface, or a combination thereof comprise at least one not collapsible material. Not collapsible materials, such as those described herein, hold their shape under storage conditions, and in use conditions, for a device of the present invention. It is desired that a device of the present invention not fold in upon itself while in sure or when stored, particularly when in use as a device of the present invention would function for longer periods of time when not collapsible materials are used. A majority of the materials discussed herein would be considered not collapsible, inclusive of a majority of plastics, polymers, and other such materials,

In a further aspect of the present invention, the first planar surface, the second planar surface, or a combination thereof have the same texture, different texture, or a combination thereof. In some instances, the surface of a device of the present invention can be smooth, and in other instances the surface of a device of the present invention can be textured. Smooth surfaces can be more comfortable for the subject, for example. Rough or textured surfaces can, for example, enhance the ability of a device of the present invention to stay in place during operation.

In an additional aspect of the present invention, the first planar surface, the second planar surface, or a combination thereof include cushioned material, not cushioned material, or a combination thereof. Cushioned materials have been discussed herein and can include silicone, rubber and other compressible materials such as hydrogels and as they are known in the art. Cushioned materials can be desirable for the comfort of the user.

In another aspect of the present invention, the first planar surface, the second planar surface, or a combination thereof include a unitary structure, a non-unitary structure, or a combination thereof. A device of the present invention can be provided, and used, as a single unit or as multiple units.

### B. Shape of Device

In another aspect of the present invention, the solid, thin, and planar structure is generally triangular, generally square, generally polygonal, generally circular, generally oval or a combination thereof, in shape. In general, a device of the present invention can be of any shape and size that is appropriate. These are but a few of the possible configurations of a device of the present invention. The size of a device of the present invention can be in the range of about 30 mm×about 30 mm with a thickness of about 1.0 mm; and about 45 mm×about 55 mm with a thickness of about 2.0 mm. Preferred sizes are about 35 mm×about 40 mm with a thickness of about 1.5 mm; and about 38 mm×about 44 mm with a thickness of about 1.5 mm. These sizes are preferable for a human subject having a normal sized buccal cavity, and can be changed as appropriate or needed for a human subject of different sized buccal cavity or for a non-human subject.

In a further aspect of the present invention, the solid, thin, and planar structure has rounded corners. Rounded corners, when corners are present, would tend to provide enhanced comfort for the subject when a device of the present invention is in use. Thus, this configuration is a preferred aspect of the present invention.

In an additional aspect of the present invention, the solid, thin, and planar structure has one or more through holes, indentations, partial through holes, or a combination thereof to allow release of components within said device, allow fluids to pass through said device, enhance the comfort of a device of the present invention, assist in said device placement and staying in place, or a combination thereof. Through holes can be made by molding, drilling, cutting, laminating, and the like and provide through space so that saliva and other fluids can readily pass through a device of the present invention to add comfort for the subject when a device of the present invention is in place, and can enhance the release of materials such as medicament when present within or on a device of the present invention. Partial through holes, like dimples on a golf ball, can be made in the same way as through holes. These partial through holes can be used to expose internal layers of a device of the present invention, such as those including medicaments that can be released from a device of the present invention.

### C. Medicament

In another aspect of the present invention, a device of the present invention includes at least one medicament in a pharmaceutically effective amount to treat a disease, disorder or condition of a subject when said device operably engaged in the buccal cavity of said subject. Any appropriate medicament can be used. Preferably, the medicament is used to treat wounds of the mouth, particularly those in contact with or in close proximity to a device of the present invention when in use with a subject, but that need not be the case.

In a further aspect of the present invention, the medicament is within said device, on the surface of said device, or a combination thereof. A medicament can be provided in an appropriate amount within a device of the present invention, such as in a drug containing layer internal to a device of the present invention, or can be provided in an outer layer of a device of the present invention, or throughout a device of the present invention. Alternatively, an additional structure, such as a tab or strip of drug containing material can be used. Such tabs or strips can be readily dissolvable or slowly dissolvable while in use with a subject.

In an additional aspect of the present invention, the medicament comprises a drug, a therapeutic, a remedy, a soothing agent, a pain reliever, an anti-inflammatory, and anti-infective, and anti-bacterial, and antibiotic, and anti-viral, an anti-fungal, anti-yeast, or a combination thereof. This list of medicaments is not limiting and relates to the types of diseases, disorders, and conditions that can be expected in the buccal cavity of a subject when a device of the present invention is in use. For example, soothing agents can be used to treat cold sores or tooth aches, and anti-yeast agents can be used to treat thrush and other conditions. The selection of the agents and dose thereof can be a design choice of the skilled artisan, particularly looking towards the dose, route of administration, and regime of administration. The positioning of a device of the present invention adjacent to or in close proximity to the locus of disease, disorder, or condition of the subject can reduce the required overall dose provided by a device of the present invention and released by a device of the present invention while in operation.

In a further aspect of the present invention, the medicament can be any medicament to treat a subject for any disease, disorder, or condition, wherein the medicament is administered to the subject through absorption through the buccal cavity, by swallowing, or a combination thereof. The choice of medicament, disease, disorder, or condition, dose, dose, regime, route of administration, and other pharmacological variable is generally one of design choice of one skill in the art.

In another aspect of the present invention, a device of the present invention is operably engaged in the buccal cavity of a subject, said device releases a pharmaceutically effective amount of said medicament to be absorbed by the oral mucosa, to be swallowed, or a combination thereof. The medicament released by a device of the present invention can be intended to be delivered to the point of contact of a device of the present invention within the buccal cavity, areas in close proximity to a device of the present invention, the entire buccal cavity, to be absorbed through the mucosa. In the alternative, the medicament can be intended to swallowed and delivered to the gastrointestinal tract. Or, a combination of absorption and swallowing. Drugs absorbed through the oral mucosa, including sublingual, does not pass directly to the liver for detoxification of the drug as is the case when drugs are absorbed in the digestive tract, and thus in the instance of absorption within the buccal cavity, less drug can be expected to be administered.

In a further aspect of the present invention, the medicament treats a disease, disorder, or condition of said subject. The selection and choice of drug or drugs to be administered by way of a device of the present invention, the dose thereof, the route of administration, the regime, and other routine pharmacological factors depends on the disease, disorder, or condition of the subject, and the locus of that disease, disorder, or condition and related pharmacological considerations, being within the skill of the ordinary artisan. The variables and factors are combined to provide an efficacious treatment for a disease, disorder, or condition of a subject using a device of the present invention while operably engaged with a subject.

### D. Subject

In another aspect of the present invention, the subject can be a mammal, a non-human primate, or a human. Preferably the subject is a human, being of any age, preferably having at least one tooth.

### V. A Method of Making a Medicated Inner Cheek Pad

The present invention includes as a fifth aspect a method of making a medicated inner cheek pad device of the present invention, comprising:
a) in the instance of molding manufacture, comprising the following steps:
   i) provide moldable polymer;
   ii) providing at least one medicament;
   ii) molding said device; and
   iii) optionally performing finishing for said device;
b) in the instance of laminating manufacture, comprising the following steps:
   i) provide multiple sheets of material;
   ii) providing at least one medicament;
   ii) laminating the multiple sheets of material together for form a laminate;
   iii) optionally cutting out or otherwise removing devices from said laminate; and
   iv) optionally performing finishing for said device; and
c) in the instance of single layer manufacture, comprising the following steps:
   i) providing a sheet of material;
   ii) providing at least one medicament;
   ii) cutting out or otherwise removing devices from said sheet of material; and
   iii) optionally performing finishing for said device;
d) a combination of a), b), and c);
wherein a device of the present invention is produced.

In general, a device of the present invention can be made using any appropriate materials, any appropriate means, and any appropriate methods of manufacture as are known in the art or later developed. Some of those materials, means, and methods are described herein.

In one aspect of the present invention, a device of the present invention can be made in whole or in part by 3D printing. 3D printing is known in the art, and the section of materials, methods and the like to manufacture a device of the present invention can be readily accomplished based on the state of the art.

In another aspect of the present invention, the molding in a) above includes injection molding. Injection molding is well known in the art, and the section of materials, methods and the like to manufacture a device of the present invention can be readily accomplished based on the state of the art.

In a further aspect of the present invention, the laminating in b) above includes heat welding, melting, partial melting, sonic welding, adhesives, or a combination thereof. These methods of adhering surfaces together, such as but not limited to those made of plastic or polymer, are well known in the art, and the section of materials, methods and the like to manufacture a device of the present invention can be readily accomplished based on the state of the art.

In another aspect of the present invention, the cutting out or otherwise removing devices in b) and c) above includes cutting devices, cutting methods, scissors, razors, dyes, cutting dyes, or a combination thereof. These methods of cutting materials, such as but not limited to those made of plastic or polymer, are well known in the art, and the section of materials, methods and the like to manufacture a device of the present invention can be readily accomplished based on the state of the art.

In an additional aspect of the present invention, the finishing in a), b), and c) above includes smoothing or roughening of surfaces, corners, edges, or a combination thereof. When considering the comfort of a user of a device of the present invention, the surfaces, corners, and edges of a device of the present invention can preferably be smooth. In order to have a device of the present invention stay in place while in use, those surfaces, corners, and edges may be roughened or remain rough. Smoothing techniques such as polishing are known in the art, such as by buffing or otherwise polishing by chemical means, mechanical means, or a combination thereof as they are known in the art Surface roughening can be likewise accomplished by chemical means, mechanical means, or a combination thereof as they are known in the art.

In another aspect of the present invention, the finishing in a), b), and c) above includes rounding of surfaces, corners, edges, or a combination thereof. When considering the comfort of a user of a device of the present invention, the surfaces, corners, and edges of a device of the present invention can preferably be rounded. Rounding techniques such as polishing are known in the art, such as by buffing or otherwise polishing by chemical means, mechanical means, or a combination thereof as they are known in the art.

In a further aspect of the present invention, the finishing in a), b), and c) above includes adding at least one additional component to a core of a device of the present invention. Additional components, for example, can be layers of material can be added to a device of the present invention, and can be made of the same or different materials as the core device, such as by lamination or adhesion. The additional component can be the same shape and size as a core device of the present invention, or can be smaller or larger, and thus fit in size and/or dimension, be larger in size and/or dimension, smaller in size and/or dimension, or a combination thereof.

In an additional aspect of the present invention, the at least one additional component comprises at least one medicament. In one preferred aspect of the present invention, the additional component includes a drug. Further, the drug can provided in an additional layer. The additional layer with a drug can be, for example, in a tab form of dissolvable material, resorbable material, degradable material, or a combination thereof that releases drug when operably engaged in the buccal cavity of a subject when in operation. Or, the drug can be released from a layer that does not dissolve, resorb, or degrade in the buccal cavity of a subject when in operation.

### VI. A Method of Using a Medicated Inner Cheek Pad

The present invention includes as a sixth aspect a method of using a medicated inner cheek pad device of the present invention, comprising:

a) providing a device of the present invention;

b) providing a subject in need of a device of the present invention; and

c) operably engaging said device of the present invention within the buccal cavity of said subject so that said at least one drug is provided to said subject in a pharmaceutically active amount to treat a disease, disorder, or condition of said subject.

These aspects of the present invention, along with others, are described in detail herein. Generally, a device of the present invention when in use can prevent or treat biting of the inner surface of the cheek, prevent or treat other injury to the inner surface of the cheek, or a combination thereof. In addition, a device of the present invention can provide at least one medicament to the locus of such injury, or injury within the buccal cavity of the subject, or can be a route of administration for a medicament to be absorbed by the buccal cavity of said subject, inclusive of sub lingual, being swallowed to be absorbed by the gastro intestinal tract, or a combination thereof. Other benefits of the use of a device of the present invention are also provided and described here, or are otherwise provided.

In one preferred embodiment of the present invention, the is wherein the device of the present invention prevents the subject from injuring the inner surface of the cheek of the subject when operably engaged in the mouth of the subject, pediatric or adult, when the subject has dental appliances, namely orthodontic braces and the like, engaged with the outer surface of the teeth of the subject. In this instance, such braces are routinely damaged and sharp edges and points result that protrude and poke or otherwise damage the inside of the mouth, particularly the inner cheek of the user. Braces tend to be in place in a subject for about a year or more, and the instance of such breakage is essentially 100%. Currently, the wearer of braces is provided wax or other material to place over the broken braces, to use when such breakage occurs, which is not a very viable option. A device of the present invention can be provided to wearers of braces for use when discomfort from normal usage of braces, to prevent injury to the cheek or other areas of the mouth such as while taking part in sports or other activities including sleeping and such where braces can irritate the mouth, and when braces break and can and do result in injury to the inner cheek.

### EXAMPLES

### Example 1: Protective Inner Cheek Pad

This example establishes a preferred protective inner cheek pad device, method of making such a device, and method of using such a device.

### Device and Method of Making

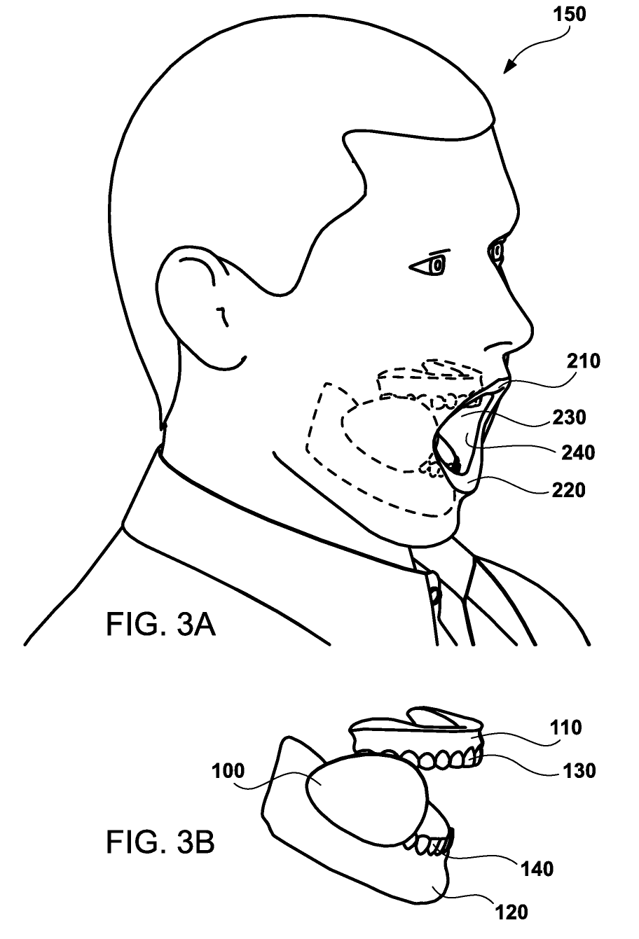

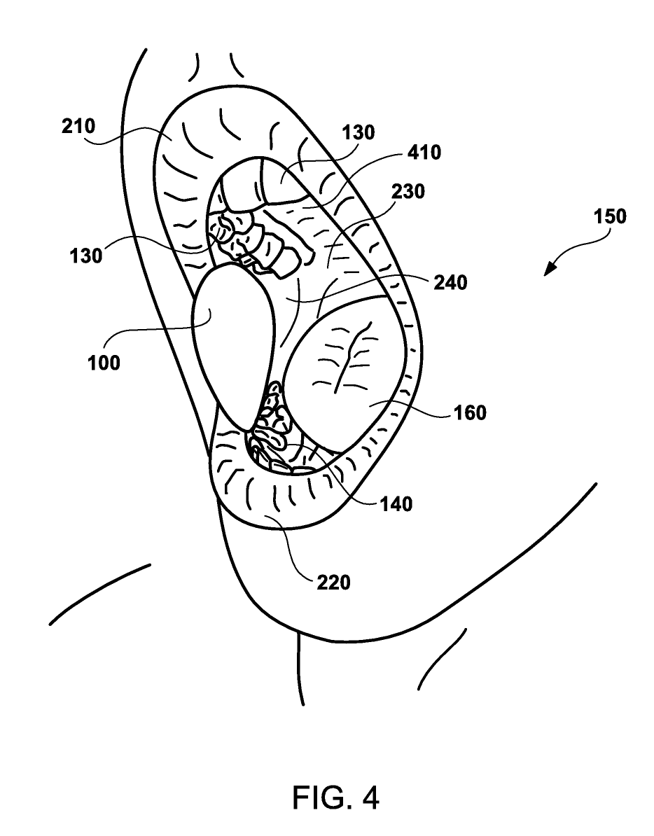

In one preferred aspect of the present invention, a device of the present invention is in the shape of round-cornered triangle or a rounded-tip pear shape (similar to the shape of a guitar pick) FIG. 3 or a short oval shape. FIG. 4. Although other pad shapes, essentially any shape, can be in included in the present invention, the physiology of the inner cheeks and teeth can in some ways influence an optimal shape of a preferred device of the present invention.

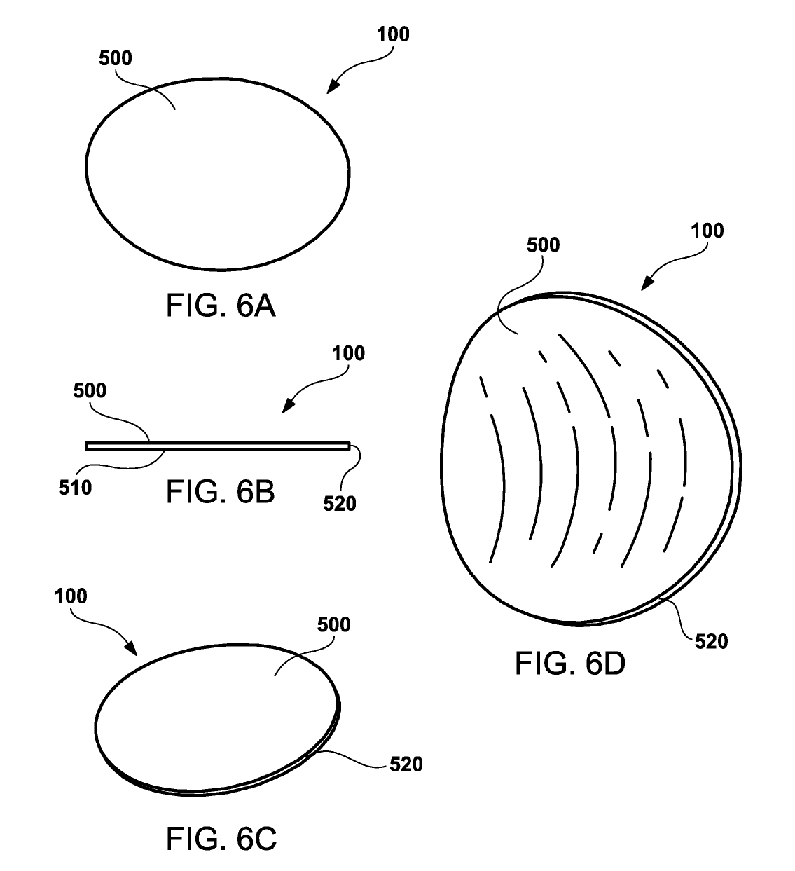

There are two general preferred sizes of a device of the present invention, generally small size and large size. A small size device (FIG. 6) being generally suitable for children, youth, and people with small mouth and smaller oral configuration. A larger size (FIG. 3 and FIG. 4) is preferred for adults with large mouth and larger oral configuration. Of course, other pad sizes may be used. The size of a device of the present invention can be in the range of about 30 mm×about 30 mm with a thickness of about 1.0 mm; and about 45 mm×about 55 mm with a thickness of about 2.0 mm. Preferred sizes are about 35 mm×about 40 mm with a thickness of about 1.5 mm; and about 38 mm×about 44 mm with a thickness of about 1.5 mm. These sizes are preferable for a human subject having a normal sized buccal cavity, and can be changed as appropriate or needed for a human subject of different sized buccal cavity or for a non-human subject.

The preferred measurements of a device of the present invention can preferably have length and width measurements of the inner cheek pad can have a tolerance of about 1 mm. The pad thickness is preferably about to be 1.5 mm; however for ease of manufacturing, a standard material thickness of 1/16 inch can be used as that thickness is readily obtainable. Preferably, devices of the present invention are made by cutting such as using a cutting dye from sheets of suitable material such as those described herein, such as but not limited to of non-toxic natural rubber, class 6 medical grade silicone, or food grade silicone.

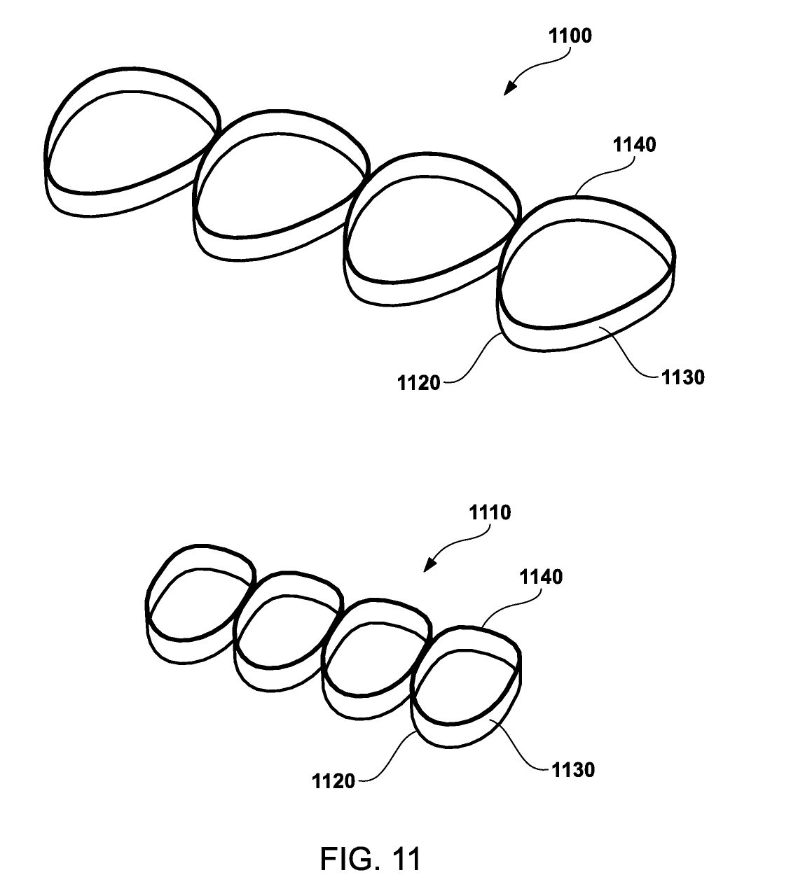

Preferably, a device of the present invention can be made of non-toxic natural rubber, class 6 medical grade silicone, or food grade silicone. Such materials being readily available in sheet form, such as from Diversified Silicone Products, Santa Fe Springs, Calif. Other materials may be found to be suitable to use as well, such as but not limited to natural rubber, latex, or a combination thereof. Generally, care should be taken when silicone is cut by heat based methods such as laser or heat/melting dye cuts as silicone emits a toxic gas when heated. In such instances, the use of mechanical force cutting using dyes can be preferable, such as die-press cutting using appropriate dyes such as cutting edge dyes. Dyes can be manufactured my any number of outlets, one being Superior in South Bay, Calif. Dyes are depicted in FIG. 11.

In some cases, a cloth-like or a paper-like material of various thicknesses and hardness can be preferable, such as but not limited to the absorption of liquid. However, this need not be the case.

The inner cheek can and are preferably trimmed to fit an individual user, such as after being cut from sheets of medical or food grade silicone. Trimming can essentially be done anywhere by anyone and in some instances to have a device of the present invention be custom fit to an individual user.

Preferably, at least one side of the Inner Cheek Pad is to be smooth so that it will sit against the teeth yet it will not impede the movement of the teeth. In some instances, at least one side can be soft and/or cushiony like a sponge so that it feels comfortable against the inner cheek's cut or sore. However, for ease of manufacturing, both sides of a device of the present invention can have the same texture. Devices made of commercially available sheets of food-grade silicone, both sides had the same texture. In addition, preferred hardness being about 50 to about 60 duro silicone (duro is the measure of hardness of the silicone), where suppliers are readily available and identified herein.

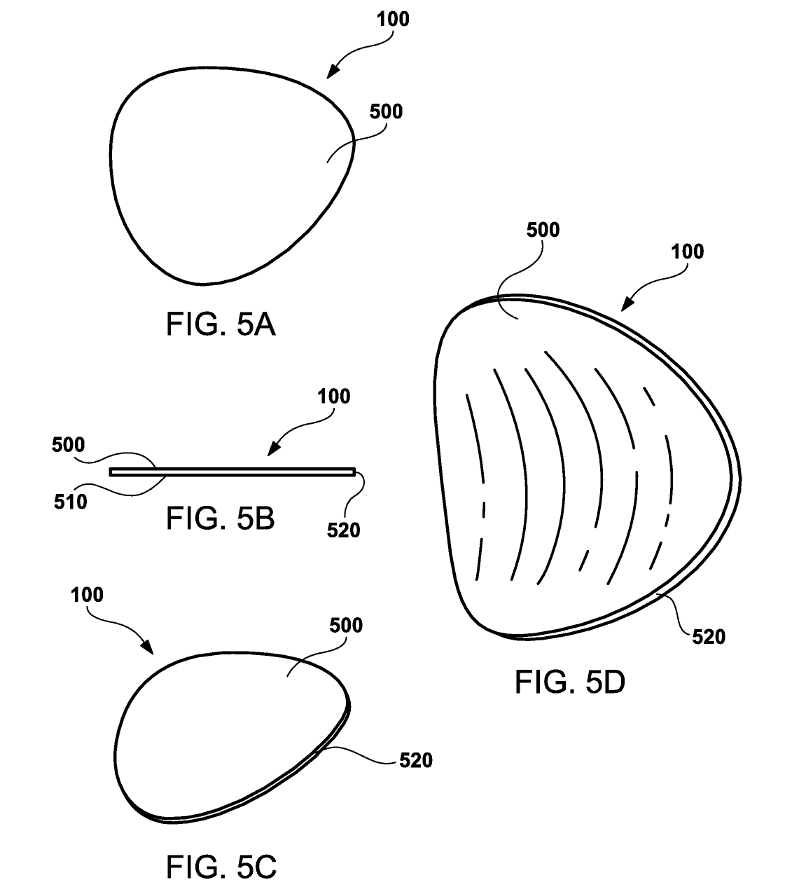

In the case of the medicated version of a device of the present invention, the medicine can be in the form of a dry film and can be placed on one or both sides of the pad. This medication becomes activated by the wetness of the saliva while placed against the inner cheek (FIG. 5).

A device of the present invention can be flavored as well. Flavoring can be provided within the device, on the surface of the device, on an additional layer of the device, or a combination thereof, such as what can be used for the introduction of a medicament into or onto a device of the present invention (spray on, tabs, layers, or a combination thereof as examples). Preferably, this aspect of the present invention can be used for children who have just had dental work done and their cheeks are still numbed. The flavor encourages the child subject and keeps them motivated to keep a device of the present invention in their mouth for a longer period until the cheek and gums' numbness is subsided and sensitivity is regained. Another use for the flavor would be to signal the user that they have regained feeling in the cheeks and gums once they are able to taste the flavor. This would be the signal to remove a device of the present invention under certain conditions of use, such as after dental or mouth procedures that result in numbing and the device being used to prevent self-biting.

### Use of Device

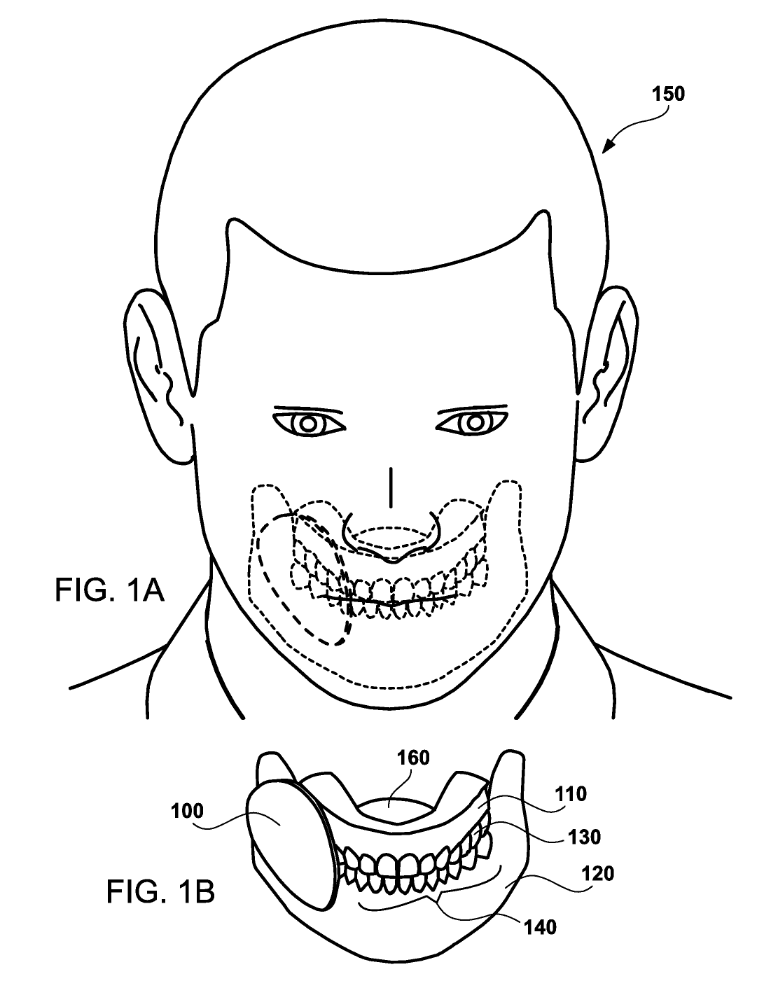

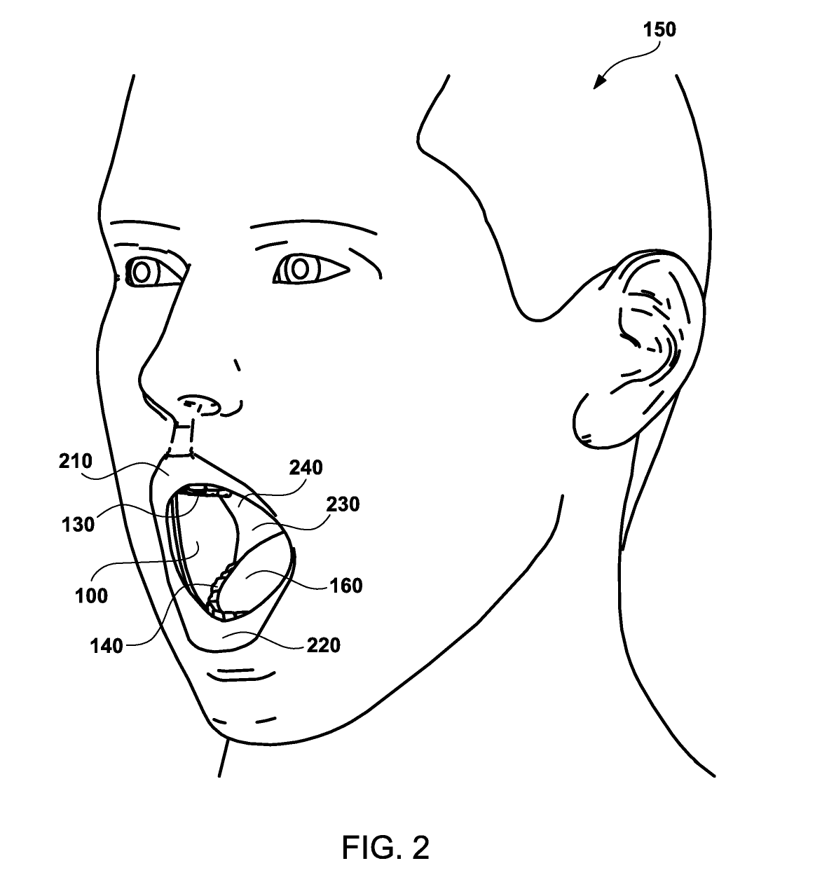

There are many uses of a device of the present invention. A few are described below and herein.
A. A device of the present invention being preferably a cushioned (non-collapsing) pad to be placed between the affected cheeks and the teeth in order to prevent the cheeks from being bitten repeatedly and allow the healing process of already damaged tissues to be quicker (FIG. 1 and FIG. 2).
B. A device of the present invention can be used to prevent this accidental biting such as but not limited to after numbing medical procedures of the mouth are performed, whereby a device of the present invention can be temporarily placed between the teeth and the inner cheeks until normal feeling is regained in the inner cheek area.
C. A device of the present invention being preferably a soft or cushioned (non-collapsing) pad to be placed between the affected cheeks and the broken or dislodged braces or other in mouth medical devices in order to prevent the cheeks from being injured by sharp points of the metal braces.

When in use, a device of the present invention is preferably placed in the inner part of the mouth between the cheek and teeth (FIG. 1 and FIG, 2). The device of the present invention preferably does not impede the speech and will not come out of place unless pulled out intentionally.

As with other inter-oral devices or substances, there can be certain warnings for use in order. These include in some instances not sleeping with a device of the present invention engaged in the mouth to avoid choking hazards. There can be some design ideas for an inner cheek pad that may be suited for overnight use. These can include a string or a band to be placed outside the mouth while attached to the pad in order to prevent accidental swallowing of the pad or accidental choking on the pad.

If a subject is prone to or sensitive to gagging, a device of the present invention may not be suitable for some. Relaxing exercises and peer or adult supervision can gradually help with gagging sensitivity problems. Eating or drinking with using a device of the present invention is not recommended in most instances. However, a device of the present invention can be temporarily removed from the mouth and then put back in its place.

A device of the present invention is suitable for the general public. However, it is recommended that children consult with a dentist or an orthodontist before using them and use of a device of the present invention in children be under the supervision of an adult.

Preferably, it is recommended to use an antiseptic mouthwash to rinse mouth before and after using a device of the present invention. Also, general good hygiene practice such as washing hands before arid after handling the inner cheek pads.

### Example 2: Medicated Inner Cheek Pad

This example establishes a preferred medicated inner cheek pad device, method of making such a device, and method of using such a device.

### Device and Method of Making

In one preferred aspect of the present invention, a device of the present invention is in the shape of round-cornered triangle or a rounded-tip pear shape (similar to the shape of a guitar pick) FIG. 3 or a short oval shape. FIG. 4. Although other pad shapes, essentially any shape, can be in included in the present invention, the physiology of the inner cheeks and teeth can in some ways influence an optimal shape of a preferred device of the present invention.

There are two general preferred sizes of a device of the present invention, generally small size and large size. A small size device (FIG. 6) being generally suitable for children, youth, and people with small mouth and smaller oral configuration. A larger size (FIG. 3 and FIG. 4) is preferred for adults with large mouth and larger oral configuration. Of course, other pad sizes may be used. The size of a device of the present invention can be in the range of about 30 mm×about 30 mm with a thickness of about 1.0 mm; and about 45 mm×about 55 mm with a thickness of about 2.0 mm. Preferred sizes are about 35 mm×about 40 mm. with a thickness of about 1.5 mm; and about 38 mm×about 44 mm with a thickness of about 1.5 mm. These sizes are preferable for a human subject having a normal sized buccal cavity, and can be changed as appropriate or needed for a human subject of different sized buccal cavity or for a non-human subject.

The preferred measurements of a device of the present invention can preferably have length and width measurements of the inner cheek pad can have a tolerance of about 1 mm. The pad thickness is preferably about to be 1.5 mm; however for ease of manufacturing, a standard material thickness of 1/16 inch can be used as that thickness is readily obtainable. Preferably, devices of the present invention are made by cutting such as using a cutting dye from sheets of suitable material such as those described herein, such as but not limited to of non-toxic natural rubber, class 6 medical grade silicone, or food grade silicone,.

Preferably, a device of the present invention can be made of non-toxic natural rubber, class 6 medical grade silicone, or food grade silicone. Such materials being readily available in sheet form, where suppliers are readily available and identified herein. Other materials may be found to be suitable to use as well.

In some cases, a cloth-like or a paper-like material of various thicknesses and hardness can be preferable, such as but not limited to the absorption of liquid. However, this need not be the case.

The inner cheek can and are preferably trimmed to fit an individual user, such as after being cut from sheets of medical or food grade silicone. Trimming can essentially be done anywhere by anyone and in some instances to have a device of the present invention be custom fit to an individual user.

Preferably, at least one side of the Inner Cheek Pad is to be smooth so that it will sit against the teeth yet it will not impede the movement of the teeth. In some instances, at least one side can be soft and/or cushiony like a sponge so that it feels comfortable against the inner cheek's cut or sore. However, for ease of manufacturing, both sides of a device of the present invention can have the same texture. Devices made of commercially available sheets of food-grade silicone, both sides had the same texture. In addition, preferred hardness being about 50 to about 60 duro silicone (duro is the measure of hardness of the silicone) where suppliers are readily available and identified herein.

In certain situations, medications are needed to be administered thru saliva in the cheeks as opposed to being ingested dim the mouth and stomach (the digestive system). Some examples of situations where drug delivery through the oral route of administration is problematic include, but are not limited to the following: people who have a hard time swallowing pills; patients with anxiety issues which cause phobia of swallowing pills; patients with acute gag reflex which can cause vomiting; patients with certain liver conditions; patients with various digestive system problems; following a stroke or surgery or gastroesophageal reflux; patients with neurological disorders (multiple sclerosis, Parkinson's disease); or a combination thereof.

An inner cheek pad of the present invention can assist in buccal delivery of medication by providing a medium that can sit against the cheeks while containing a medicated film on one or both sides.

Nearly any type of medicament can be used in a device of the present invention, including but not limited to: cardiovascular drugs (Nitroglycerin, Verapamil); steroids; barbiturates; enzymes; vitamins; medications for mental health conditions; opioid dependence treatment (Buprenorphine and Naloxone) and prevention of withdrawal symptoms; antibiotics, anti-bacterial; anti-viral; anti-fungal; anti-yeast; weight control, weight loss; cancer; numbing agents; soothing agents; menthol; or a combination thereof.

In the case of the medicated version of a device of the present invention, the medicine can be in the form of a dry film and can be placed on one or both sides of the pad. This medication becomes activated by the wetness of the saliva while placed against the inner cheek (FIG. 5). Furthermore, the medicament can be integrated within the device as a whole, or otherwise provided on the surface of the device, in whole or in part, such as but not limited to by a coating such as, for example, by soaking or spray coating. Additional coatings on a device of the present invention can also provide a medicament. One or more medicaments can be so provided.

Any medicament can be used, preferably those that are absorbed through the surfaces of the buccal cavity, and those that can be swallowed, or a combination thereof. Thus, at least two routes of administration can be provided by the device of the present invention. The dose of the medicament is a choice of the skilled artisan, along with the dosage of the medicament released in total and over time. Thus, the medicament, route of administration, dose, and regime of administration of one or more medicaments is provided by a device of the present invention. Other pharmacological and pharmacokinetic considerations can be taken into account by those skilled in the art.

A device of the present invention can be flavored as well. Flavoring can be provided within the device, on the surface of the device, on an additional layer of the device, or a combination thereof, such as what can be used for the introduction of a medicament into or onto a device of the present invention (spray on, tabs, layers, or a combination thereof as examples). Preferably, this aspect of the present invention can be used for children who have just had dental work done and their cheeks are still numbed. The flavor encourages the child subject and keeps them motivated to keep a device of the present invention in their mouth for a longer period until the cheek and gums' numbness is subsided and sensitivity is regained. Another use for the flavor would be to signal the user that they have regained feeling in the cheeks and gums once they are able to taste the flavor. This would be the signal to remove a device of the present invention under certain conditions of use, such as after dental or mouth procedures that result in numbing and the device being used to prevent self-biting.

### Use of Device

There are many uses of a device of the present invention. A few are described herein. When in use, a device of the present invention is preferably placed in the inner part of the mouth between the cheek and teeth (FIG. 1 and FIG. 2). The device of the present invention preferably does not impede the speech and will not come out of place unless pulled out intentionally.

As with other inter-oral devices or substances, there can be certain warnings for use in order. These include in some instances not sleeping with a device of the present invention engaged in the mouth to avoid choking hazards. There can be some design ideas for an inner cheek pad that may be suited for overnight use. These can include a string or a band to be placed outside the mouth while attached to the pad in order to prevent accidental swallowing of the pad or accidental choking on the pad.

If a subject is prone to or sensitive to gagging, a device of the present invention may not be suitable for some. Relaxing exercises and peer or adult supervision can gradually help with gagging sensitivity problems.

Eating or drinking with using a device of the present invention is not recommended in most instances. However, a device of the present invention can be temporarily removed from the mouth and then put back in its place.

A device of the present invention is suitable for the general public. However, it is recommended that children consult with a dentist or an orthodontist before using them and use of a device of the present invention in children be under the supervision of an adult.

Preferably, it is recommended to use an antiseptic mouthwash to rinse mouth before and after using a device of the present invention. Also, general good hygiene practice such as washing hands before and after handling the inner cheek pads.

All publications, including patent documents and scientific articles, referred to in this application and the bibliography and attachments are incorporated by reference in their entirety for all purposes to the same extent as if each individual publication were individually incorporated by reference.

All headings and titles are for the convenience of the reader and should not be used to limit the meaning of the text that follows the heading, unless so specified.

What is claimed is:

1. A protective inner cheek pad device, comprising:
a) a flexible, thin, and planar structure configured to fit between the outer surface of the teeth of a subject and the inner surface of the cheek of a subject when in use in a subject;
b) said solid, thin, and planar structure having two planar surfaces and a circumference surface;
c) a first planar surface that is not liquid absorbent and can operably engage the outer surface that is of the teeth of a subject;
d) a second planar surface that is not liquid absorbent and can operably engage the inner surface of the cheek of a subject; and
wherein said inner cheek pad device prevents said subject from biting said inner surface of the cheek of said subject when operably engaged in the mouth of said subject; and
wherein said inner cheek pad device prevents said subject from injuring said inner surface of the cheek of said subject when operably engaged in the mouth of said subject when the subject has dental appliances engaged with said outer surface of the teeth of a subject.

2. The protective inner cheek pad device of claim 1,
wherein said flexible, thin, and planar structure is a laminated structure, a not laminated structure, or a combination thereon.

3. The protective inner cheek pad device of claim 1;
wherein said first planar surface and said second planar surface are made of the same material, different material, or a combination thereof.

4. The protective inner cheek pad device of claim 1;
wherein said first planar surface, said second planar surface, or a combination thereof comprise non-toxic material.

5. The protective inner cheek pad device of claim 1;
wherein said first planar surface, said second planar surface, or a combination thereof comprise solid material, semi-solid material, or a combination thereof.

6. The protective inner cheek pad device of claim 1;
wherein said first planar surface, said second planar surface, or a combination thereof comprise plastic, rubber, polymer, or a combination thereof.

7. The protective inner cheek pad device of claim 1;
wherein said first planar surface, said second planar surface, or a combination thereof comprise silicone, medical grade silicone, food grade silicone, natural rubber, or a combination thereof.

8. The protective inner cheek pad device of claim 1;
wherein said first planar surface, said second planar surface, or a combination thereof comprise at least one not collapsible material.

9. The protective inner cheek pad device of claim 1;
wherein said first planar surface, said second planar surface, or a combination thereof have the same texture, different texture, or a combination thereof.

10. The protective inner cheek pad device of claim 1;
wherein said first planar surface, said second planar surface, or a combination thereof comprise cushioned material, not cushioned material or a combination thereof.

11. The protective inner cheek pad device of claim 1,
wherein said first planar surface, said second planar surface, or a combination thereof comprise a unitary structure, a non-unitary structure, or a combination thereof.

12. The protective inner cheek pad device of claim 1,
wherein said solid, thin, and planar structure is generally triangular, generally square, generally polygonal, generally circular, generally oval or a combination thereof, in shape.

13. The protective inner cheek pad device of claim 1,
wherein said solid, thin, and planar structure has rounded corners.

14. The protective inner cheek pad device of claim 1,
wherein said solid, thin, and planar structure has one or more through holes, indentations, partial through holes, or a combination thereof to allow release of components within said device, allow fluids to pass through said device, enhance the comfort of the device, assist in said device placement and staying in place, or a combination thereof.

15. The protective inner cheek pad device of claim 1,
wherein said device comprises at least one medicament in a pharmaceutically effective amount to treat a disease, disorder or condition of a subject when said device operably engaged in the buccal cavity of said subject.

16. The protective inner cheek pad device of claim 15,
wherein said medicament is within said device, on the surface of said device, or a combination thereof.

17. The protective inner cheek pad device of claim 15,
wherein said medicament comprises a drug, a therapeutic, a remedy, a soothing agent, a pain reliever, an anti-inflammatory, and anti-infective, and anti-bacterial, and antibiotic, and anti-viral, an anti-fungal, anti-yeast, or a combination thereof.

18. The protective inner cheek pad device of claim 15,
wherein when said device is operably engaged in the buccal cavity of a subject, said device releases a pharmaceutically effective amount of said medicament to be absorbed by the oral mucosa, to be swallowed, or a combination thereof.

19. The protective inner cheek pad device of claim 15,
wherein said medicament treats a disease, disorder, or condition of said subject.

20. A method of using a protective inner cheek pad device of claim 1, comprising:
a) providing a device of claim 1;
b) providing a subject in need of a device of claim 1; and
c) operable engaging said device of claim 1 within the buccal cavity of said subject;
wherein said subject obtains the benefits of the device of claim 1.

21. The method of claim 20, wherein said benefits of the device of claim 1 comprise:
a) wherein the device of claim 1 prevents said subject from biting said inner surface of the cheek of said subject when operably engaged in the mouth of said subject;
b) wherein the device of claim 1 prevents said subject from injuring said inner surface of the cheek of said subject when operably engaged in the mouth of said subject when the subject has dental appliances engaged with said outer surface of the teeth of a subject;
c) wherein the device of claim 1 provides medicaments to treat one or more wounds of the mouth; and
d) a combination thereof.
